(19) United States
(12) Patent Application Publication
(10) Pub. No.: US 2025/0249266 A1
(43) Pub. Date: Aug. 7, 2025

(54) STIMULATION FIELD MODEL MANIPULATION

(71) Applicant: Boston Scientific Neuromodulation Corporation, Valencia, CA (US)

(72) Inventor: Leon Mauricio Juarez Paz, Berlin (DE)

(21) Appl. No.: 19/024,853

(22) Filed: Jan. 16, 2025

Related U.S. Application Data
(60) Provisional application No. 63/549,974, filed on Feb. 5, 2024.

(51) Int. Cl. A61N 1/372; A61N 1/02; A61N 1/36

(52) U.S. Cl. CPC A61N 1/37247; A61N 1/025; A61N 1/36185

(57) ABSTRACT

Systems and methods for programming a neuromodulation therapy to a neuromodulation device are disclosed. An exemplary system includes an electrostimulator to provide electrostimulation to a neural target, and a programmer device operable by a user to program the electrostimulator. The programmer device includes a graphical user interface (GUI), and a controller circuit to controllably display on the GUI the neural target, and receive a user input to create or modify a graphical stimulation field representation with respect to the neural target. Based on the graphical stimulation field representation, the controller circuit can estimate a stimulation setting, including determining a stimulation test volume, activated by an application of the estimated stimulation setting, that substantially matches the received graphical stimulation field representation. The electrostimulator can deliver electrostimulation energy in accordance with the estimated stimulation setting.

### CLAIM OF PRIORITY

This application claims the benefit of U.S. Provisional Application No. 63/549,974, filed on Feb. 5, 2024, which is hereby incorporated by reference in its entirety.

### TECHNICAL FIELD

This document relates generally to medical devices, and more particularly, to computer-assisted adjustment of stimulation field and programming of a medical device to provide neuromodulation therapy.

### BACKGROUND

Neuromodulation (or “neural neuromodulation”, also referred to as “neurostimulation” or “neural stimulation”) has been proposed as a therapy for a number of conditions. Often, neuromodulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS systems have been used as a therapeutic modality for the treatment of chronic pain syndromes. PNS has been used to treat chronic pain syndrome and incontinence, with a number of other applications under investigation. FES systems have been applied to restore some functionality to paralyzed extremities in spinal cord injury patients. DBS can be used to treat a variety of diseases or disorders.

Stimulation systems, such as implantable electrostimulators, have been developed to provide therapy for a variety of treatments. An implantable electrostimulator can include a pulse generator and one or more leads each including a plurality of stimulation electrodes. The stimulation electrodes are in contact with or near target tissue to be stimulated, such as nerves, muscles, or other tissue. The control module generates a control signal to the pulse generator, which generates electrostimulation pulses that are delivered by the electrodes to the target tissue in accordance with an electrode configuration and a set of stimulation parameters.

Neuromodulation therapy can be programmed into an electrostimulator by a user (e.g., a clinician or an authorized device user) using a programmer device. In an example of DBS, visually guided programming has been used to reduce the time needed by clinicians to find optimal or desired stimulation settings for the patient to improve clinical benefits and/or to reduce therapy side effects. However, the stimulation settings generally involve a large number of stimulation parameters and various combinations thereof, and some of such parameter combinations may not be readily intuitive for clinicians. This makes programming of a stimulation device for a patient a challenging task.

### SUMMARY

Programming an electrostimulator using a programmer device generally requires a user to select or provide values for a number of therapy parameters such as amplitude, frequency, pulse width, waveforms, electrodes (cathode and anode) selected for delivering electrostimulation energy. The programming may also involve selecting, or providing values for, a sensing parameter for sensing physiological data from the patient, such as a neural activity signal or an electrophysiological signal. Sensing a physiological signal while providing an electrostimulation therapy provides a feedback mechanism that may help assess patient responses to the therapy, which may help regulate or optimize a closed-loop therapy. However, finding an optimal or desired closed-loop therapy for the patient and programming such therapy to the electrostimulator generally require testing of multiple device parameters (e.g., sensing parameter and therapy parameters) and various combinations thereof, which can be time consuming and add significant complexity. Moreover, as more advanced neuromodulation therapies are developed and made available to the patient, the level of sophistication involved in neuromodulation therapy testing, optimization, and programming continues to rise. Some users (e.g., clinicians or authorized device users) may not be familiar with, thus do not use or know how to best use, certain device features pertaining to advanced neural activity signal sensing and processing, feature extraction and selection, and neuromodulation therapy programming. Even with proper training, some users may not readily envision how a different programmed value of a device feature would affect the therapeutic outcome in a patient. Additionally, although some neuromodulation systems are capable of automatically recommending stimulation settings for a patient such as based on user specification of stimulating target volume and/or avoidance volume, some recommended combinations of stimulation parameters are impossible or difficult to test clinically, and/or not intuitive to clinicians (e.g., to readily understand the therapeutic effects and/or side effects). As a result, some advanced device features may be significantly underused if not misused. Some users may not consistently customize their programming workflow to search for individualized optimal therapies. The process of searching for and programming an optimal therapy for the patient can be inefficient. Furthermore, the longer the user stays within a given programming session, the more battery power is consumed, especially when testing and optimization of certain programmable parameters requires active communication between the electrostimulator system (e.g., battery-powered implantable device) and the programmer device.

The present inventors have recognized an unmet need for apparatus and techniques to assist programming of neuromodulation therapy with more intuitive control elements and programming tools, thereby improving programming efficiency and effectiveness. According to one embodiment, an electrostimulation system comprises an electrostimulator to provide electrostimulation to a neural target of a patient via at least one lead, and a programmer device operable by a user to program the electrostimulator. The programmer device includes a graphical user interface (GUI), and a controller circuit to control the GUI to display a graphical representation of the neural target, receive a user input to create or modify a graphical stimulation field representation with respect to the neural target, and estimate a stimulation setting using a computational model based on the graphical stimulation field representation. The estimation includes, via an iterative process, determining a stimulation test volume, activated by an application of the estimated stimulation setting, that substantially matches the received graphical stimulation field representation. The electrostimulator can deliver electrostimulation energy in accordance with the estimated stimulation setting.

Various examples discussed in this document may improve the efficiency and quality of testing and programming neuromodulation therapy to a patient. In an example of programming an implantable neuromodulation device for DBS, embodiments described in the present document would allow a clinician to intuitively manipulate the stimulation field via a graphical user interface, such as moving the stimulation field towards target stimulation volumes and away from avoidance volumes, or defining or modifying the stimulation field size and shape, while the corresponding stimulation settings (e.g., stimulation parameters or electrode configurations) can be automatically determined (and updated as the user modifies the stimulation field) in the background using a computational model. The apparatus and methods described herein may expedite the search process for an individualized optimal neuromodulation therapy for the patient, reduce therapy programming time for users of different levels of experience with the device features, improve their work efficiency, and reduce overall operation cost. The reduced programming time may also reduce battery power consumption and extend battery life for battery-powered neuromodulation devices.

Example 1 is a neuromodulation system that includes: an electrostimulator configured to provide electrostimulation to a neural target in a patient via at least one lead; and a programmer device operable by a user to program the electrostimulator, the programmer device including: a graphical user interface (GUI); and a controller circuit configured to: display on the GUI a graphical representation of the neural target; receive, via the GUI, a user input to create or modify a graphical stimulation field representation with respect to the neural target; based on the received user input of the graphical stimulation field representation, estimate a stimulation setting including one or more stimulation parameters or electrode configurations using a computational model, the estimation including determining a stimulation test volume, activated by an application of the estimated stimulation setting, that substantially matches the received graphical stimulation field representation; and generate a control signal to the electrostimulator to deliver electrostimulation energy to the neural target in accordance with the estimated stimulation setting.

In Example 2, the subject matter of Example 1 optionally includes the computational model that can include an inverse modeling algorithm relating a stimulation volume to a stimulation setting.

In Example 3, the subject matter of any one or more of Examples 1-2 optionally includes the controller circuit that can be configured to display on the GUI a graphical representation of a base stimulation field model, wherein the user input to create or modify the graphical stimulation field representation is further with respect to the base stimulation field model.

In Example 4, the subject matter of any one or more of Examples 1-3 optionally includes the GUI that can include one or more user interface control elements operable by the user to provide the user input to create or modify the graphical stimulation field representation.

In Example 5, the subject matter of Example 4 optionally includes the user input to create or modify the graphical stimulation field representation that can include, via the one or more user interface control elements: creating an initial graphical stimulation field representation; and modifying the initial graphical stimulation field representation, including one or more of translating, rotating, resizing, flipping, or cropping of the initial graphical stimulation field representation or a portion thereof.

In Example 6, the subject matter of Example 5 optionally includes, wherein modifying the initial graphical stimulation field representation includes resizing or reshaping the initial graphical stimulation field representation or a portion thereof via a drag-and-drop user interface control element from the GUI.

In Example 7, the subject matter of any one or more of Examples 1-6 optionally includes the user input to create or modify the graphical stimulation field representation spatially relative to the at least one lead.

In Example 8, the subject matter of Example 7 optionally includes the estimated stimulation setting that can include an electrode configuration specifying a selection of one or more active electrodes from the at least one lead and a fractionalization of electrical current flowing through the selected one or more active electrodes.

In Example 9, the subject matter of any one or more of Examples 1-8 optionally includes the estimated stimulation setting that can include one or more of an amplitude, a pulse width, or a frequency of electrostimulation pulses.

In Example 10, the subject matter of any one or more of Examples 1-9 optionally include the controller circuit that can include configured to create an initial graphical stimulation field representation based on a user selection of a stimulation target volume within or proximate the neural target.

In Example 11, the subject matter of any one or more of Examples 1-10 optionally includes the controller circuit that can be configured to create an initial graphical stimulation field representation based on a user selection of a stimulation avoidance volume within or proximate the neural target.

In Example 12, the subject matter of any one or more of Examples 1-11 optionally include the controller circuit that can be configured to create an initial graphical stimulation field representation based on a user input of a base stimulation setting.

In Example 13, the subject matter of any one or more of Examples 1-12 optionally includes the controller circuit that can be further configured to: determine a reference graphical stimulation field representation corresponding to the stimulation test volume; and display on the GUI the reference graphical stimulation field representation overlaid upon the graphical stimulation field representation received as the user input.

In Example 14, the subject matter of Example 13 optionally includes the controller circuit that can be further configured to: present on the GUI an indication of a difference between the reference graphical stimulation field representation and the graphical stimulation field representation received as the user input; and receive a user acceptance or rejection of the received graphical stimulation field representation.

In Example 15, the subject matter of Example 14 optionally includes the controller circuit that can be configured to: in response to the user acceptance of the received graphical stimulation field representation, generate the control signal to the electrostimulator to deliver electrostimulation energy in accordance with the estimated stimulation setting; and in response to the user rejection of the received graphical stimulation field representation, prompt the user to further modify the received stimulation field model on the GUI.

Example 16 is a method of operating a programmer device to program an electrostimulator to deliver electrostimulation energy to a neural target in a patient via at least one lead. The method includes steps of: displaying, on a graphical user interface (GUI) associated with the programmer device, a graphical representation of the neural target; receiving, via the GUI, a user input to create or modify a graphical stimulation field representation with respect to the neural target; based on the received user input of the graphical stimulation field representation, estimating a stimulation setting including one or more stimulation parameters or electrode configurations using a computational model, the estimation including determining a stimulation test volume, activated by an application of the estimated stimulation setting, that substantially matches the received graphical stimulation field representation; and generating a control signal to the electrostimulator to deliver the electrostimulation energy to the neural target in accordance with the estimated stimulation setting.

In Example 17, the subject matter of Example 16 optionally includes the computational model that can include an inverse modeling algorithm relating a stimulation volume to a stimulation setting.

In Example 18, the subject matter of any one or more of Examples 16-17 optionally includes displaying on the GUI a graphical representation of a base stimulation field model, wherein the user input to create or modify the graphical stimulation field representation is further with respect to the base stimulation field model.

In Example 19, the subject matter of any one or more of Examples 16-18 optionally includes the user input to create or modify the graphical stimulation field representation that can include, via one or more user interface control elements in the GUI operable by the user: creating an initial graphical stimulation field representation; and modifying the initial graphical stimulation field representation, including one or more of translating, rotating, resizing, flipping, or cropping of the initial graphical stimulation field representation or a portion thereof.

In Example 20, the subject matter of any one or more of Examples 16-19 optionally includes the user input to create or modify the graphical stimulation field representation that can be spatially relative to the at least one lead, wherein the estimated stimulation setting includes an electrode configuration specifying a selection of one or more active electrodes from the at least one lead and a fractionalization of electrical current flowing through the selected one or more active electrodes.

In Example 21, the subject matter of any one or more of Examples 16-20 optionally includes receiving the user input to create or modify the graphical stimulation field representation that can include: receiving a user selection of one or more of a stimulation target volume or a stimulation avoidance volume each within or proximate the neural target; and automatically creating an initial graphical stimulation field representation based on the user selection of the one or more of a stimulation target volume or the stimulation avoidance volume.

In Example 22, the subject matter of any one or more of Examples 16-21 optionally includes receiving the user input to create or modify the graphical stimulation field representation that can include: receiving a user input of a base stimulation setting; and automatically creating an initial graphical stimulation field representation based on the base stimulation setting.

In Example 23, the subject matter of any one or more of Examples 16-22 optionally includes: determining a reference graphical stimulation field representation corresponding to the stimulation test volume; presenting on the GUI an indication of a difference between the reference graphical stimulation field representation and the graphical stimulation field representation received as the user input; receiving a user acceptance or rejection of the received graphical stimulation field representation; in response to the user acceptance of the received graphical stimulation field representation, generating the control signal to the electrostimulator to deliver electrostimulation energy in accordance with the estimated stimulation setting; and in response to the user rejection of the received graphical stimulation field representation, prompting the user to further modify the received stimulation field model on the GUI.

The description that follows will generally focus on the use of the invention within a DBS systems. However, the present invention may find applicability with any implantable neurostimulator device system, including SCS system, Vagus Nerve Stimulation (VNS) system, Sacral Nerve Stimulation (SNS) systems, and the like. For example, apparatus and methods for detecting (and maintaining) exceptionally small evoked neural activities as described herein can be used to detect evoked neural activities in closed-loop DBS therapy, or therapees of other regions of the nervous system. The following examples illustrate various aspects of the examples described herein.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of a neurostimulation system.

FIG. 2 illustrates an example of a stimulation device and a lead system, such as may be implemented in the neurostimulation system of FIG. 1.

FIG. 3 illustrates an example of a programming device, such as may be implemented in the neurostimulation system of FIG. 1.

FIG. 4 illustrates an example of an implantable pulse generator (IPG) and an implantable lead system, such as an example implementation of the stimulation device and lead system of FIG. 2.

FIG. 5 illustrates an example of an IPG and an implantable lead system, such as the IPG and lead system of FIG. 4, arranged to provide neurostimulation to a patient.

FIG. 6 illustrates an example of portions of a neurostimulation system.

FIG. 7 illustrates an example of an implantable stimulator and one or more leads of an implantable neurostimulation system, such as the implantable neurostimulation system of FIG. 6.

FIG. 8 illustrates an example of an external programming device of an implantable neurostimulation system, such as the implantable neurostimulation system of FIG. 6.

FIG. 9 illustrates an example of a user interface of an external programming device, such as the external programming device of FIG. 8.

FIGS. 10A-10B illustrate an example of a GUI that receives a user input to create or modify a graphical stimulation field representation, and presents corresponding stimulation settings automatically determined based on the graphical stimulation field representation.

FIGS. 11A-11B illustrate another example of a GUI that receives a user input to create or modify a graphical stimulation field representation, and presents corresponding stimulation settings automatically determined based on the graphical stimulation field representation.

FIG. 12 is a flow chart illustrating and example method for programming an electrostimulator to provide electrostimulation to a patient.

FIG. 13 illustrates generally a block diagram of an example machine upon which any one or more of the techniques (e.g., methodologies) discussed herein may perform.

### DETAILED DESCRIPTION

This document describes systems and methods for programming neuromodulation therapy to a neuromodulation device. An exemplary system comprises an electrostimulator to provide electrostimulation to a neural target of a patient via at least one lead, and a programmer device operable by a user to program the electrostimulator. The programmer device includes a graphical user interface (GUI), and a controller circuit to control the GUI to display a graphical representation of the neural target, receive a user input to create or modify a graphical stimulation field representation with respect to the neural target, and estimate a stimulation setting using a computational model based on the graphical stimulation field representation. The estimation includes determining a stimulation test volume, activated by an application of the estimated stimulation setting, that substantially matches the received graphical stimulation field representation. The electrostimulator can deliver electrostimulation energy in accordance with the estimated stimulation setting.

The following detailed description of the present subject matter refers to the accompanying drawings which show, by way of illustration, specific aspects and examples in which the present subject matter may be practiced. These examples are described in sufficient detail to enable those skilled in the art to practice the present subject matter. Other examples may be utilized and structural, logical, and electrical changes may be made without departing from the scope of the present subject matter. References to “an”, “one”, or “various” examples in this disclosure are not necessarily to the same example, and such references contemplate more than one example. The following detailed description is, therefore, not to be taken in a limiting sense, and the scope is defined only by the appended claims, along with the full scope of legal equivalents to which such claims are entitled.

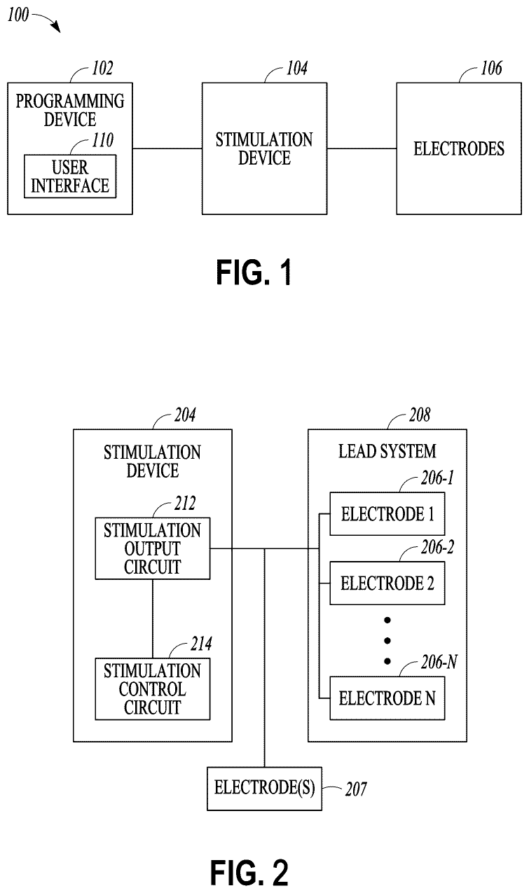

FIG. 1 illustrates an example of a neurostimulation system 100. The system 100 includes electrodes 106, a stimulation device 104, and a programming device 102. The electrodes 106 may be configured to be placed on or near one or more neural targets in a patient. The stimulation device 104 may be electrically connected to the electrodes 106 and deliver neurostimulation energy, such as in the form of electrical pulses, to the one or more neural targets though the electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters may be programmable by a user, such as a physician or other caregiver who treats the patient using the system 100. The programming device 102 may provide the user with accessibility to the user-programmable parameters. In various embodiments, the programming device 102 may be communicatively coupled to the stimulation device 104 via a wired or wireless link.

In this document, a “user” includes a physician or other clinician or caregiver who treats the patient using the system 100; a “patient” includes a person who receives or is intended to receive neurostimulation delivered using the system 100. In various embodiments as described in this document, the patient can be allowed to adjust neurostimulation using the system 100 to certain extent, such as by creating or modifying a graphical stimulation field representation on a user interface, programming certain therapy parameters, and entering feedback and clinical effect information.

In various embodiments, the programming device 102 can include a user interface 110 that allows the user to control the operation of the system 100 and monitor the performance of the system 100 as well as conditions of the patient including responses to the delivery of the neurostimulation. The user can control the operation of the system 100 by setting and/or adjusting values of the user-programmable parameters.

In various embodiments, the user interface 110 can include a graphical user interface (GUI) that allows the user to set and/or adjust values of the user-programmable parameters by creating and/or editing graphical representations of stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In some examples, the GUI may allow the user to set and/or adjust a graphical stimulation field representation. Based on the received graphical stimulation field representation, the programming device 102 may estimate a stimulation setting (also referred to as a stimulation configuration) using a computational model. The estimated stimulation setting includes one or more stimulation parameters or electrode configurations. The estimation includes, via an iterative process, determining a stimulation test volume, activated by an application of the estimated stimulation setting, that substantially matches the received graphical stimulation field representation. In various embodiments, neurostimulation pulses for a stimulation period (such as the duration of a therapy session) may be delivered to multiple stimulation fields.

In various embodiments, the system 100 may be used in various neurostimulation applications. The user interface 110 may be configured to allow the user to control the operation of the system 100 for neurostimulation. For example, the system 100 and the user interface 110 may be configured for DBS applications. Such DBS configuration includes various features that may simplify the task of the user in programming stimulation device 104 for delivering DBS to the patient, such as the features discussed in this document.

FIG. 2 illustrates an example of a stimulation device 204 and a lead system 208, such as may be implemented in the neurostimulation system 100. The stimulation device 204 represents an embodiment of the stimulation device 104 and includes a stimulation output circuit 212 and a stimulation control circuit 214. The stimulation output circuit 212 can produce and deliver neurostimulation pulses. The stimulation control circuit 214 can control the delivery of the neurostimulation pulses from the stimulation output circuit 212 using the plurality of stimulation parameters, which specifies a pattern of the neurostimulation pulses. The lead system 208 can include one or more leads each configured to be electrically connected to the stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from the stimulation output circuit 212 through a set of electrodes selected from the electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to the stimulation device 204, such as one or more electrodes each being a portion of or otherwise incorporated onto a housing of the stimulation device 204. Monopolar stimulation uses a monopolar electrode configuration with at least one electrode selected from the electrodes 206 and at least one electrode from the electrode(s) 207. Bipolar stimulation uses a bipolar electrode configuration with two electrodes selected from the electrodes 206 and none of the electrode(s) 207. Multipolar stimulation uses a multipolar electrode configuration with multiple electrodes selected from the electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, the lead system 208 includes two leads each having eight electrodes.

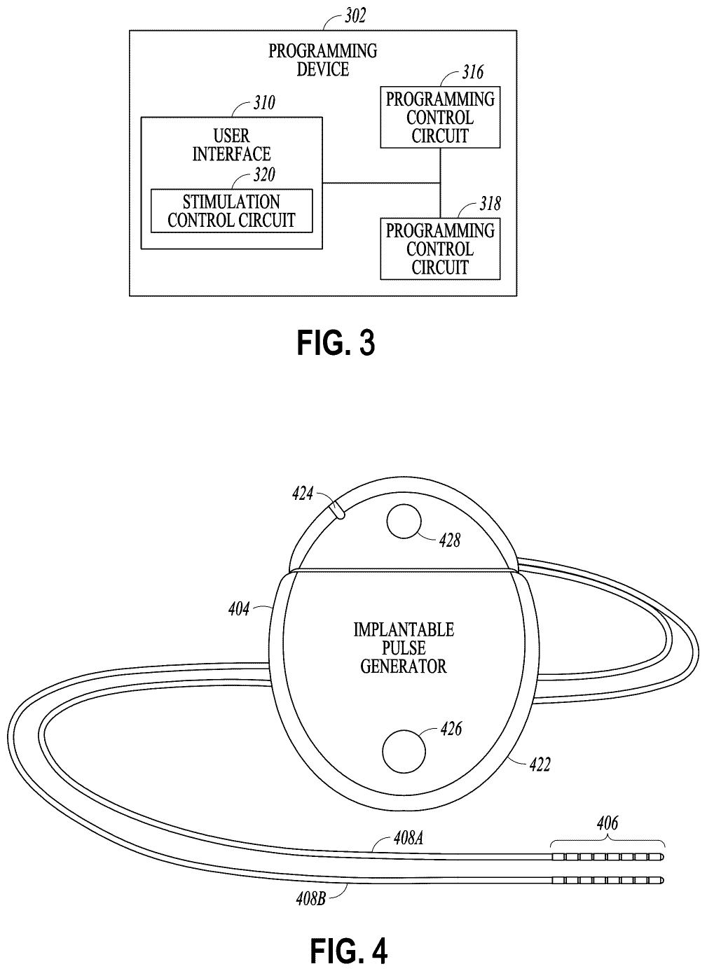

FIG. 3 illustrates an example of a programming device 302, such as may be implemented in the neurostimulation system 100. The programming device 302 represents an embodiment of the programming device 102 and includes a storage device 318, a programming control circuit 316, and a user interface 310. The programming control circuit 316 can generate the plurality of stimulation parameters that controls the delivery of the neurostimulation pulses according to a specified stimulation configuration that can define, for example, stimulation waveform or electrode configuration. The user interface 310 represents an embodiment of the user interface 110, and may include a stimulation control circuit 320. The storage device 318 stores information used by the programming control circuit 316 and the stimulation control circuit 320, such as information about a stimulation device that relates the stimulation configuration to the plurality of stimulation parameters and information relating the stimulation configuration to a volume of activation in the patient. In various embodiments, the stimulation control circuit 320 can be configured to support one or more functions allowing for programming of stimulation devices, such as the stimulation device 104 including its various embodiments as discussed in this document.

In various embodiments, the user interface 310 can allow for definition of a pattern of neurostimulation pulses for delivery during a neurostimulation therapy session by creating and/or adjusting one or more stimulation waveforms using a graphical method. The definition can also include a graphical representation of one or more stimulation fields each associated with one or more pulses in the pattern of neurostimulation pulses. As used in this document, a “stimulation configuration” can include the pattern of neurostimulation pulses including the one or more stimulation fields, or at least various aspects or parameters of the pattern of neurostimulation pulses including the one or more stimulation fields. In various embodiments, the user interface 310 includes a GUI that allows the user to define the pattern of neurostimulation pulses and perform other functions using graphical methods. As similarly described above with respect to FIG. 1, the GUI of the user interface 310 may allow the user to set and/or adjust a graphical stimulation field representation. In this document, “neurostimulation programming” can include the definition of the one or more stimulation waveforms, including the definition of one or more stimulation fields.

In various embodiments, circuits of the neurostimulation 100, including its various embodiments discussed in this document, may be implemented using a combination of hardware and software. For example, the circuit of user interface 110, the stimulation control circuit 214, the programming control circuit 316, and the stimulation control circuit 320, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an example of an implantable pulse generator (IPG) 404 and an implantable lead system 408. The IPG 404 represents an example implementation of the stimulation device 204. The lead system 408 represents an example implementation of the lead system 208. As illustrated in FIG. 4, the IPG 404 that can be coupled to implantable leads 408A and 408B at a proximal end of each lead. The distal end of each lead includes electrical contacts or electrodes 406 for contacting a tissue site targeted for electrical neurostimulation. As illustrated, the leads 408A and 408B each include eight electrodes 406 at the distal end. The number and arrangement of the leads 408A and 408B and the electrodes 406 are only an example, and other numbers and arrangements are possible. In various embodiments, the electrodes are ring electrodes. The implantable leads and electrodes may be configured by shape and size to provide electrical neurostimulation energy to a neuronal target included in the subject's brain, or configured to provide electrical neurostimulation energy to a nerve cell target included in the subject's spinal cord.

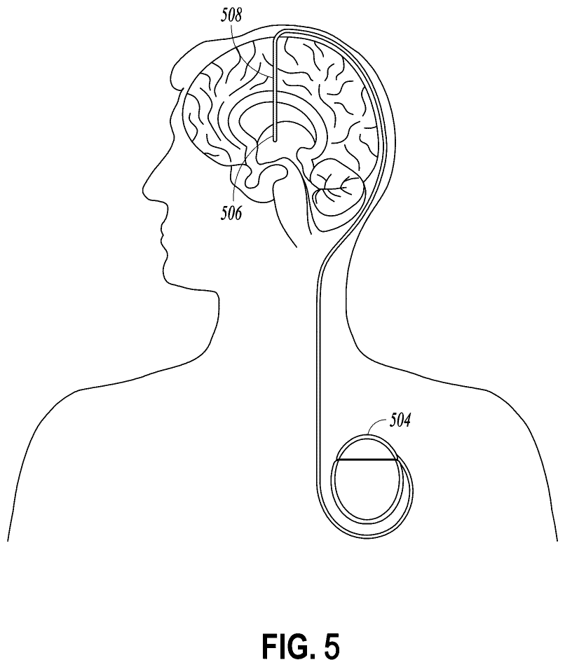

FIG. 5 illustrates an example of an IPG 504 and an implantable lead system 508 arranged to provide neurostimulation to a patient. An example of the IPG 504 includes the IPG 404. An example of the lead system 508 includes one or more of the leads 408A and 408B. In the illustrated embodiment, the implantable lead system 508 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

Returning to FIG. 4, the IPG 404 can include a hermetically-sealed IPG case 422 to house the electronic circuitry of the IPG 404. The IPG 404 can include an electrode 426 formed on IPG case 422. The IPG 404 can include an IPG header 424 for coupling the proximal ends of the leads 408A and 408B. The IPG header 424 may optionally also include an electrode 428. The electrodes 426 and/or 428 represent embodiments of the electrode(s) 207 and may each be referred to as a reference electrode. Neurostimulation energy can be delivered in a monopolar (also referred to as unipolar) mode using the electrode 426 or the electrode 428 and one or more electrodes selected from the electrodes 406. Neurostimulation energy can be delivered in a bipolar mode using a pair of electrodes of the same lead (e.g. the lead 408A or 408B). Neurostimulation energy can be delivered in an extended bipolar mode using one or more electrodes of a lead (e.g., one or more electrodes of the lead 408A) and one or more electrodes of a different lead (e.g., one or more electrodes of the lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

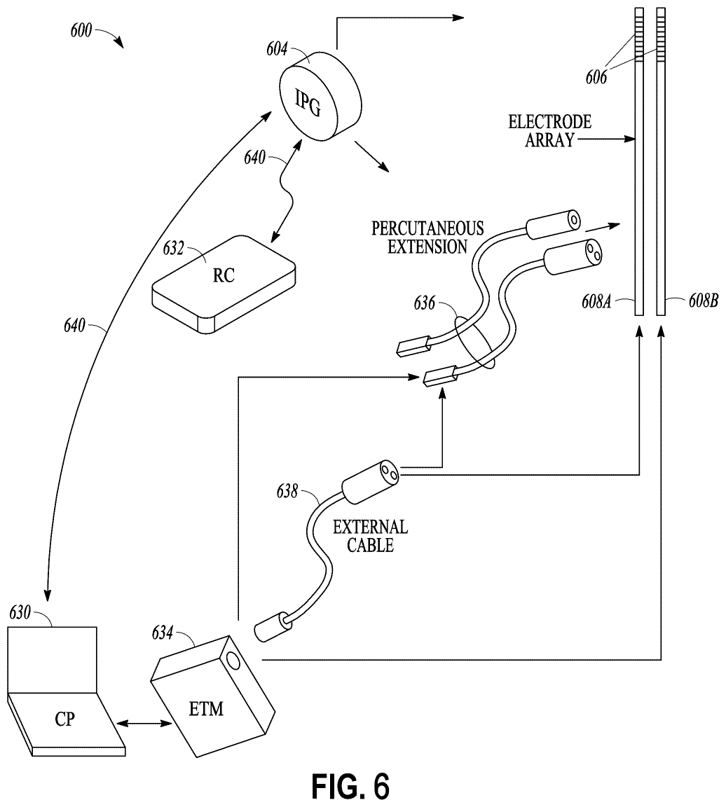

FIG. 6 illustrates an example of portions of a neurostimulation system 600. The system 600 includes an IPG 604, implantable neurostimulation leads 608A and 608B, an external remote controller (RC) 632, a clinician's programmer (CP) 630, and an external trial modulator (ETM) 634. The IPG 404 may be electrically coupled to leads 608A and 608B directly or through percutaneous extension leads 636. ETM 634 may be electrically connectable to the leads 608A and 608B via one or both of the percutaneous extension leads 636 and/or the external cable 638. The system 600 represents an embodiment of the system 100, with IPG 604 representing an embodiment of the stimulation device 104, electrodes 606 of leads 608A and 608B representing the electrodes 106, and CP 630, RC 632, and ETM 634 collectively representing the programming device 102.

The ETM 634 may be standalone or incorporated into the CP 630. The ETM 634 may have similar pulse generation circuitry as the IPG 604 to deliver neurostimulation energy according to specified modulation parameters as discussed above. The ETM 634 is an external device that is typically used as a preliminary stimulator after the leads 408A and 408B have been implanted and used prior to stimulation with the IPG 604 to test the patient's responsiveness to the stimulation that is to be provided by the IPG 604. Because the ETM 634 is external it may be more easily configurable than the IPG 604.

The CP 630 can configure the neurostimulation provided by the ETM 634. If the ETM 634 is not integrated into the CP 630, then the CP 630 may communicate with the ETM 634 using a wired connection (e.g., over a USB link) or by wireless telemetry using a wireless communications link 640. The CP 630 also communicates with the IPG 604 using a wireless communications link 640,

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. The IPG 604 can include the first coil and a communication circuit. The CP 630 can include or otherwise electrically connected to the second coil such as in the form of a wand that can be place near the IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of the IPG 604 and in the housing of the CP 630, eliminating the need for a wand or other means of inductive coupling. An example is such an RF telemetry link is a Bluetooth® wireless link.

The CP 630 can be used to set modulation parameters for the neurostimulation after the IPG 604 has been implanted. This allows the neurostimulation to be tuned if the requirements for the neurostimulation change after implantation. CP 630 can also upload information from IPG 604.

RC 632 also communicates with IPG 604 using a wireless link 340. RC 632 may be a communication device used by the user or given to the patient. The RC 632 may have reduced programming capability compared to the CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by the CP 630. The CP 630 may communicate with the RC 632 using a wired or wireless communications link. In some embodiments, the CP 630 is able to program the RC 632 when remotely located from the RC 632.

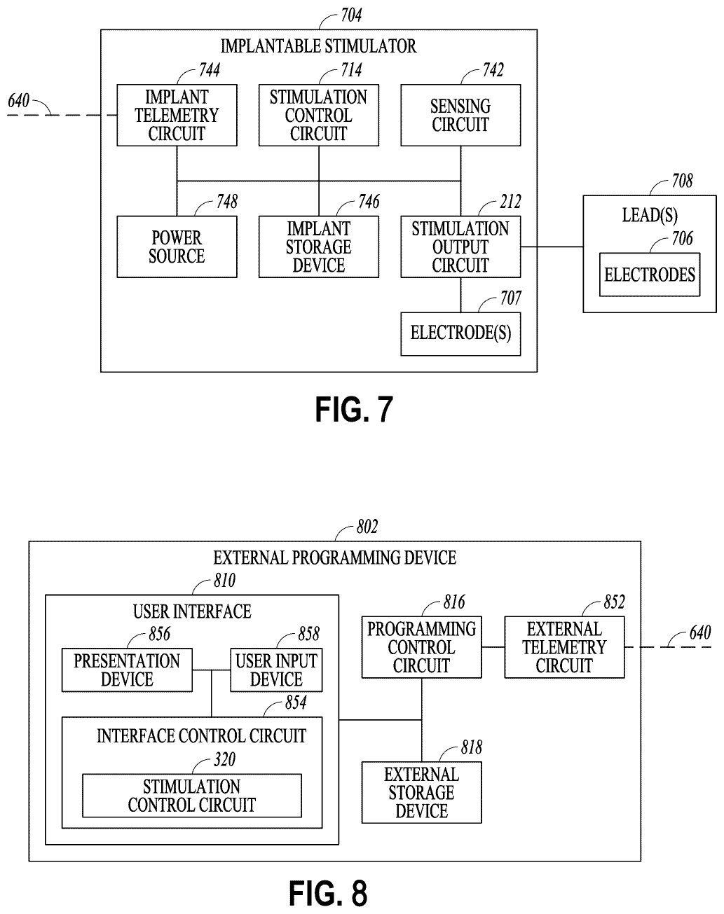

FIG. 7 illustrates an example of an implantable stimulator 704 and one or more leads 708 of an implantable neurostimulation system, such as the implantable system 600. The implantable stimulator 704 represents an embodiment of the stimulation device 104 or 204 and may be implemented, for example, as the IPG 604. The one or more lead(s) 708 represents an embodiment of lead system 208 and may be implemented, for example, as the implantable leads 608A and 608B. The one or more lead(s) 708 includes electrodes 706, which represents an embodiment of the electrodes 106 or 206 and may be implemented as electrodes 606.

The implantable stimulator 704 may include a sensing circuit 742 that is optional and required only when the stimulator needs a sensing capability, the stimulation output circuit 212, a stimulation control circuit 714, an implant storage device 746, an implant telemetry circuit 744, a power source 748, and one or more electrodes 707. The sensing circuit 742, when included and needed, senses one or more physiological signals for purposes of patient monitoring and/or feedback control of the neurostimulation. Examples of the one or more physiological signals include neural and other signals each indicative of a condition of the patient that is treated by the neurostimulation and/or a response of the patient to the delivery of the neurostimulation. The stimulation output circuit 212 is electrically connected to electrodes 706 through one or more leads 708 as well as electrodes 707, and delivers each of the neurostimulation pulses through a set of electrodes selected from the electrodes 706 and the electrode(s) 707. The stimulation control circuit 714 represents an embodiment of the stimulation control circuit 214 and controls the delivery of the neurostimulation pulses using the plurality of stimulation parameters specifying the pattern of neurostimulation pulses. In one embodiment, the stimulation control circuit 714 controls the delivery of the neurostimulation pulses using the one or more sensed physiological signals. The implant telemetry circuit 744 provides the implantable stimulator 704 with wireless communication with another device such as the CP 630 and the RC 632, including receiving values of the plurality of stimulation parameters from the other device. The implant storage device 746 stores values of the plurality of stimulation parameters. The power source 748 provides the implantable stimulator 704 with energy for its operation. In one embodiment, the power source 748 includes a battery. In one embodiment, the power source 748 includes a rechargeable battery and a battery charging circuit for charging the rechargeable battery. The implant telemetry circuit 744 may also function as a power receiver that receives power transmitted from an external device through an inductive couple. The electrode(s) 707 allow for delivery of the neurostimulation pulses in the monopolar mode. Examples of the electrode(s) 707 include the electrode 426 and the electrode 418 in the IPG 404 as illustrated in FIG. 4.

In one embodiment, implantable stimulator 704 is used as a master database. A patient implanted with the implantable stimulator 704 (such as may be implemented as the IPG 604) may therefore carry patient information needed for his or her medical care when such information is otherwise unavailable. The implant storage device 746 is configured to store such patient information. For example, the patient may be given a new RC 632 and/or travel to a new clinic where a new CP 630 is used to communicate with the device implanted in him or her. The new RC 632 and/or CP 630 can communicate with implantable stimulator 704 to retrieve the patient information stored in the implant storage device 746 through the implant telemetry circuit 744 and wireless communication link 640, and allow for any necessary adjustment of the operation of the implantable stimulator 704 based on the retrieved patient information. In various embodiments, the patient information to be stored in the implant storage device 746 may include, for example, the positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in the implant storage device 746 may include data transmitted to the implantable stimulator 704 for storage as part of the patient information and data acquired by the implantable stimulator 704, such as by using the sensing circuit 742.

In various embodiments, the sensing circuit 742 (if included), the stimulation output circuit 212, the stimulation control circuit 714, the implant telemetry circuit 744, the implant storage device 746, and the power source 748 are encapsulated in a hermetically sealed implantable housing or case, and the electrode(s) 707 are formed or otherwise incorporated onto the case. In various embodiments, lead(s) 708 are implanted such that the electrodes 706 are placed on and/or around one or more targets to which the neurostimulation pulses are to be delivered, while the implantable stimulator 704 is subcutaneously implanted and connected to the lead(s) 708 at the time of implantation.

FIG. 8 illustrates an example of an external programming device 802 of an implantable neurostimulation system, such as system 600. The external programming device 802 represents an embodiment of the programming device 102 or 302, and may be implemented, for example, as CP 630 and/or RC 632. The external programming device 802 includes an external telemetry circuit 852, an external storage device 818, a programming control circuit 816, and a user interface 810.

The external telemetry circuit 852 provides the external programming device 802 with wireless communication with another device such as the implantable stimulator 704 via the wireless communication link 640, including transmitting the plurality of stimulation parameters to the implantable stimulator 704 and receiving information including the patient data from the implantable stimulator 704. In one embodiment, external telemetry circuit 852 also transmits power to the implantable stimulator 704 through an inductive couple.

In various embodiments, the wireless communication link 640 can include an inductive telemetry link (near-field telemetry link) and/or a far-field telemetry link (RF telemetry link). For example, because DBS is often indicated for movement disorders which are assessed through patient activities, gait, balance, etc., allowing patient mobility during programming and assessment is useful. Therefore, when the system 600 is intended for applications including DBS, the wireless communication link 640 includes at least a far-field telemetry link that allows for communications between the external programming device 802 and the implantable stimulator 704 over a relative long distance, such as up to about 20 meters. The external telemetry circuit 852 and the implant telemetry circuit 744 each include an antenna and RF circuitry configured to support such wireless telemetry.

The external storage device 818 stores one or more stimulation waveforms for delivery during a neurostimulation therapy session, such as a DBS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as the implantable stimulator 704 to deliver a therapy. In various embodiments, each waveform in the one or more stimulation waveforms is definable on a pulse-by-pulse basis, and the external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g. mA) allows precise dosing of current through each specific electrode. It is suited for changing the current one contact at a time (and allows the user to do so) to shape the stimulation like a piece of clay (pushing/pulling one spot at a time).

The programming control circuit 816 represents an embodiment of the programming control circuit 316 and generates the plurality of stimulation parameters, which is to be transmitted to the implantable stimulator 704, based on a specified stimulation configuration (e.g., the pattern of neurostimulation pulses as represented by one or more stimulation waveforms and one or more stimulation fields, or at least certain aspects of the pattern). The stimulation configuration may be created and/or adjusted by the user using user interface 810 and stored in the external storage device 818. In various embodiments, programming control circuit 816 can check values of the plurality of stimulation parameters against safety rules to limit these values within constraints of the safety rules. In one embodiment, the safety rules are heuristic rules.

The user interface 810 represents an embodiment of the user interface 310 and allows the user to define the pattern of neurostimulation pulses and perform various other monitoring and programming tasks. The user interface 810 includes a presentation device 856, a user input device 858, and an interface control circuit 854. The presentation device 856 may include any type of interactive or non-interactive screens, and the user input device 858 may include any type of user input devices that supports the various functions discussed in this document, such as touchscreen, keyboard, keypad, touchpad, trackball, joystick, and mouse. In one embodiment, the user interface 810 includes a GUI. The GUI may also allow the user to perform any functions discussed in this document where graphical presentation and/or editing are suitable as may be appreciated by those skilled in the art.

The interface control circuit 854 controls the operation of the user interface 810 including responding to various inputs received by user input device 858 and defining the one or more stimulation waveforms. The interface control circuit 854 includes the stimulation control circuit 320.

In various embodiments, the external programming device 802 can have operation modes including a composition mode and a real-time programming mode. Under the composition mode (also known as the pulse pattern composition mode), the user interface 810 is activated, while the programming control circuit 816 is inactivated. The programming control circuit 816 does not dynamically updates values of the plurality of stimulation parameters in response to any change in the one or more stimulation waveforms. Under the real-time programming mode, both the user interface 810 and the programming control circuit 816 are activated. The programming control circuit 816 dynamically updates values of the plurality of stimulation parameters in response to changes in the set of one or more stimulation waveforms, and transmits the plurality of stimulation parameters with the updated values to implantable stimulator 704.

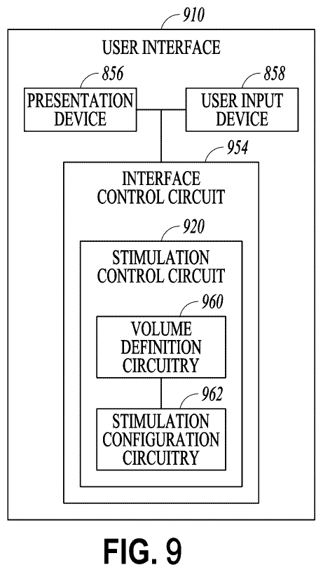

FIG. 9 illustrates an example of a user interface 910 of an external programming device, such as the external programming device 803. The user interface 910 represents an embodiment of the user interface 810, and allows the user to define a desired stimulation field and perform various other monitoring and programming tasks. The user interface 910 includes the presentation device 856, the user input device 858, and an interface control circuit 954. The presentation device 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 910 includes a GUI that allows the user to perform any functions discussed in this document where graphical presentation and/or editing are suitable as may be appreciated by those skilled in the art.

The interface control circuit 954 represents an embodiment of the interface control circuit 854 and includes a stimulation control circuit 920, which represents an embodiment of the stimulation control circuit 320 and specifies the stimulation configuration. The stimulation control circuit 920 includes volume definition circuitry 960 and stimulation configuration circuitry 962. The volume definition circuitry 960 can be configured to determine a target volume using one or more clinical effects resulting from activation of one or more test volumes by neurostimulation (e.g., delivery of the neurostimulation pulses as discussed in this document). In some embodiments, the stimulation configuration circuitry 962 can be configured to allow the user to enter or select one or more stimulation configurations each corresponding to a test volume, and configured to generate a stimulation configuration based on the target volume. In this document, a “target volume” refers to a volume of activation for which a medical device such as the implantable stimulator 704 is programmed to deliver a neurostimulation therapy to treat the patient, and a “test volume” refers to a volume of activation used in a process of determining the target volume. In one embodiment, the stimulation configuration circuitry 962 generates the stimulation configuration for activating a stimulation volume substantially matching the target volume. The target volume includes a first portion of tissue of the patient. The stimulation volume includes a second portion of the tissue. Ideally, the first portion of the tissue and the second portion of the tissue are the same portion of tissue. In practice, the stimulation volume should substantially match the target volume such that the difference between the first portion of the tissue and the second portion of the tissue is minimized.

In some embodiments, the stimulation configuration circuitry 962 may allow the user to set and/or adjust a graphical stimulation field representation. Based on the graphical stimulation field representation, a stimulation configuration (also referred to as a stimulation setting) may be estimated, such as using a computational model. The estimation includes determining a stimulation test volume, activated by an application of the estimated stimulation configuration, that substantially matches the received graphical stimulation field representation. The estimated stimulation configuration includes one or more stimulation parameters or electrode configurations that correspond to a coverage region substantially matching the user set or adjusted stimulation field. In an example, the stimulation configuration may include information about fractionalization of stimulation current or energy across multiple active electrodes.

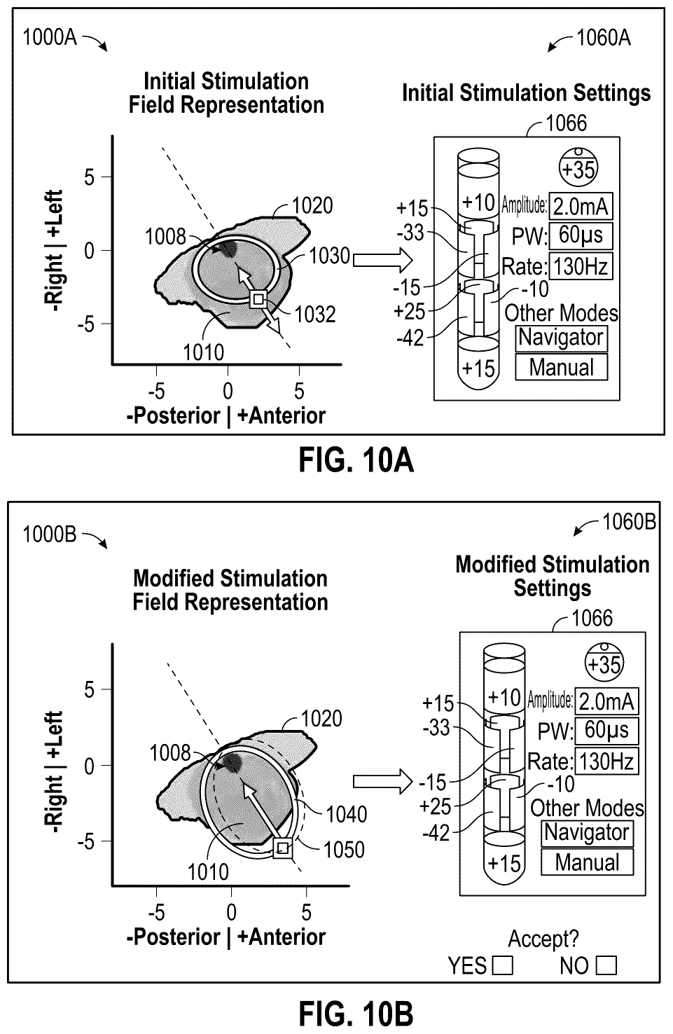

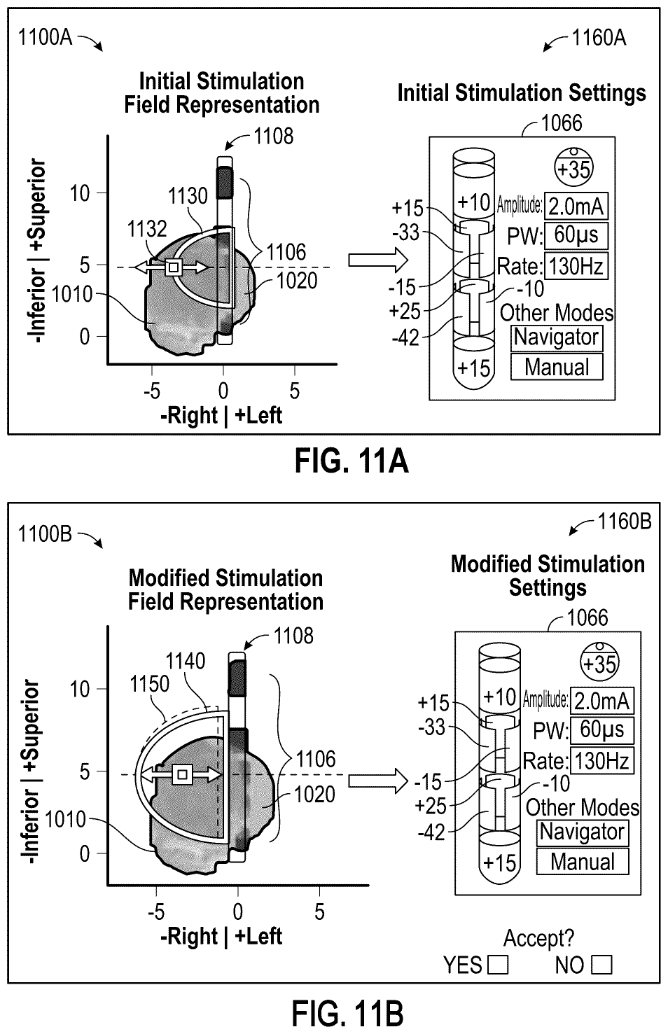

FIGS. 10A-10B and 11A-11B illustrate examples of GUI that receives a user input to create or modify a graphical stimulation field representation, and presents corresponding stimulation settings automatically determined based on the graphical stimulation field representation. The GUI can be an embodiment of the user interface 910. The volume definition circuitry 960 can present a graphical representation of a base stimulation field model (SFM) 1010 and a graphical representation of a “default” target neural structure 1020 on the GUI using the presentation device 856. The base stimulation field model (SFM) 1010 defines a therapeutic region where the stimulation thereof would generally lead to clinically acceptable therapeutic effects (e.g., high therapeutic benefits such as symptom relief, and/or low side effects). The base SFM 1010 can be a “population-based” model in that can be based on implantation data obtained from a patient population. In an example, the base SFM 1010 can be an aggregation of several individual SFMs previously created and tested to lead to clinically acceptable therapeutic effects. The default target neural structure 1020 may be a clinically acceptable therapeutic neural structure, such as Subthalamic Nucleus (STN) which is generally believed to be a DBS target for alleviating or rectifying motor symptoms of Parkinson's Disease (PD), among other movement disorders. The default target neural structure (e.g., STN) can be individually identified and localized from the patient during a pre-implantation imaging study. In practice, due to anatomical and functional differences across patients, the actual stimulation field selected and projected to produce desired therapeutic effects may not be identical to the default target neural structure 1020, and may be adjusted based on the aggregated SFM 1010. For example, only a sub-region of the target neural structure 1020 may be selected and projected to produce desired therapeutic outcomes.

The volume definition circuitry 960 can further present a graphical stimulation field representation received as a user input from the GUI. The user may create or modify the graphical stimulation field representation using one or more user interface (UI) elements based on one or more of the population-based, aggregated SFM 1010, or the individualized (i.e., patient-specific) target neural structure 1020, both of which may serve as reference volumes to assist the user in creating an initial graphical stimulation field representation. FIG. 10A illustrates a stimulation field panel 1000A where a user may create an initial graphical stimulation field representation 1030 relative to the aggregated SFM 1010 and/or the individualized target neural structure 1020. FIG. 11A illustrates a stimulation field panel 1100A where a user may create an initial graphical stimulation field representation 1130 relative to the aggregated SFM 1010 and/or the individualized target neural structure 1020. Proper UI control elements may be used to create (e.g., directly draw, or select from a bank of stored pre-defined shapes) respective initial graphical stimulation field representations 1030 and 1130. The initial graphical stimulation field representations 1030 and 1130 each have user-defined shape and size, and are located at user-defined positions with respect to the population-based aggregated SFM 1010 and/or the individualized target neural structure 1020. The initial graphical stimulation field representations 1030 and 1130 may each represent a user designated target area intended to be stimulated in one example, a user designated avoidance region not intended to be stimulated in another example, or both in a further example.

In some examples, information about the lead system being used for delivering electrostimulation and associated electrodes thereon (such as one or more of leads 408A, 408B, 608A, or 608B), including lead location and active electrodes, may be graphically presented on the GUI. The aggregated SFM 1010, the default target neural structure 1020, and the user-provided initial graphical stimulation field representation 1030 or 1130 may be presented in a two-dimensional view (e.g., a cross-sectional view of a tissue volume), as illustrated in the stimulation field panels 1000A and 1100A. Alternatively, the aggregated SFM 1010, the default target neural structure 1020, and the user-provided initial graphical stimulation field representation 1030 or 1130 may be presented in a three-dimensional space. In an example, the user may use UI control elements to change the viewing plane or the viewing angle, such as relative to the lead orientation. For example, FIGS. 10A-10B illustrate a viewing plane perpendicular to lead 1008 (shown in a top view). FIGS. 11A-11B illustrate another viewing plane in parallel to lead 1108 (shown in a side view), where the associated electrodes 1106 (an embodiment of the electrodes 406 or 606) become visible on the display. In an example, the lead 1008 and the lead 1108 represent the same lead, and the user-provided initial graphical stimulation field representations 1030 and 1130 represent the same stimulation field representation displayed on different viewing planes or different viewing angles. The graphical presentations of various objects (e.g., the aggregated SFM 1010, the default target neural structure 1020, and the initial graphical stimulation field representation 1030 and 1130) on the stimulation field panels 1000A and 1100A represent projections of the corresponding three-dimensional objects onto different viewing planes.

The user may provide the initial graphical stimulation field representation at a location spatially relative to the lead location or locations of one or more electrodes as displayed on the GUI. For example, FIG. 10A shows an initial graphical stimulation field representation 1030 covering a subregion of the aggregated SFM 1010 and the default target neural structure 1020 along and proximate to the lead 1008. FIG. 11A shows an initial graphical stimulation field representation 1130 covering a subregion of the aggregated SFM 1010 and the default target neural structure 1020 along and proximate the lead 1108.

The stimulation configuration circuitry 962 may estimate a stimulation configuration (also referred to as a stimulation setting, S) based on the received graphical stimulation field representation (M) 1030 or 1130 using a computational model (f). In an example, the computational model (f) includes an inverse modeling algorithm that automatically estimates, through an iterative process, a test volume of tissue in the patient, activated by the stimulation configuration, that substantially matches a target volume represented by the graphical stimulation field representation (M). The “substantial” match requirement is satisfied when a difference between the target volume and the “final” test volume, which is to be resulted from delivery of neurostimulation using the estimated stimulation configuration (S), falls within a specific margin, at which point the iteration process can be terminated. Such estimation process can be conceptually represented by a formula S=f(M). The target volume can be refined during the iterations using the one or more clinical effects resulting from the test volume used in each iteration. Alternatively, the user may specify a test volume for each iteration. For example, stimulation configuration circuitry 962 may receive a stimulation configuration from the user (who manually defines the stimulation configuration or selects one from stored stimulation configurations), and generate a test volume to be result from delivery of neurostimulation using the stimulation configuration. In one embodiment, the inverse modeling algorithm is based on a stimulation field model (SFM) relating a stimulation configuration to a volume of activation. The stimulation configuration can be estimated using a library including data mapping volumes of activation to stimulation configurations and/or using an analytical derivation of the stimulation configuration that generates the stimulation volume. The estimated stimulation configuration (S) includes one or more stimulation parameters or electrode configurations that correspond to a coverage region (corresponding to the final test volume) substantially matching the received graphical stimulation field representation M (corresponding to the target volume). In an example, the stimulation configuration may include information about fractionalization of stimulation current or energy across multiple active electrodes. Further details of such inverse modeling algorithm are described in commonly owned U.S. patent application Ser. No. 15/902,163, entitled “Method and Apparatus For Clinical Effects-Based Targeting of Neurostimulation” and filed on Feb. 22, 2018, and U.S. patent application Ser. No. 16/219,551, entitled “Systems and Methods for Clinical Effect-Based Neurostimulation” and filed on Dec. 13, 2018, the description of which is hereby incorporated by reference in their entirety.

The stimulation configuration circuitry 962 can present the estimated stimulation configuration (S) using presentation device 856. As illustrated in FIGS. 10A and 11A, the estimated stimulation configurations can be presented in respective stimulation configuration panels 1060A and 1160A on the display screen. The stimulation configuration panel shows electrode configuration including polarity (shown as “+” for anode and “−” for cathode) and fractionalization (e.g., percentage of stimulation current, such as +10 for 10% current applied to an anode, −33 for 33% of current applied to a cathode), and stimulation pulse parameters including amplitude, pulse width (PW), and stimulation rate or frequency. A “Navigator” button can open a navigation console that allows the user to navigate predefined stimulation configurations and select a predefined stimulation configuration. A “Manual” button can open a manual programming console that allows the user to manually define a stimulation configuration.

The user may modify the initial graphical stimulation field representation using one or more UI control elements. The modification may include, for example, one or more of translating, rotating, resizing, flipping, or cropping of the initial graphical stimulation field representation or a portion thereof. FIG. 10B illustrates a stimulation field panel 1000B where a user may modify the initial graphical stimulation field representation 1030. FIG. 11B illustrates a stimulation field panel 1100B where a user may modify the initial graphical stimulation field representation 1130. In the illustrated examples, the user may use a drag-and-drop UI control element to change the size and/or the shape of the initial graphical stimulation field representations 1030 and 1130 into respective modified graphical stimulation field representations 1040 and 1140, such as by mouse pressing and dragging an edge portion 1032 or 1132 of the initial graphical stimulation field representation, and releasing the mouse when a desired size and shape is obtained. In accordance with the modification of the graphical stimulation field representation (M), the stimulation configuration circuitry 962 may update the estimated stimulation configuration (S) using the estimation formula S=f(M) applied to the modified graphical stimulation field representation. The stimulation configuration circuitry 962 can present the updated estimate of stimulation configuration (S) in respective stimulation configuration panels 1060B and 1160B on the display screen, as illustrated in FIGS. 10B and 11B.

As stated above, the inverse modeling algorithm f determines the estimated stimulation configuration (S) through an iterative process that stops when the “final” test volume to be resulted from delivery of neurostimulation according to the estimated stimulation configuration (S) substantially matches the target volume represented by the graphical stimulation field representation (M), such as when a difference therebetween falls within a specific margin. The “final” test volume thus determined generally sufficiently approximates, but may not be identical to, the target volume. Such difference may be due to implementation restrictions on stimulation configuration, such as value ranges of certain stimulation parameters or combinations of stimulation parameters. The stimulation configuration circuitry 962 may determine a reference graphical stimulation field representation (M′) 1050 corresponding to the “final” test volume, which may be substantially close to, but not identical to, the user-provided graphical stimulation field representation (M) 1040. The stimulation configuration circuitry 962 may present on the GUI an indication of a difference between the graphical stimulation field representation (M′) 1050 and the user-provided graphical stimulation field representation (M) 1040, and prompt the user to accept or reject the graphical stimulation field representation 1040, as illustrated in FIG. 10B. In response to the user acceptance of the graphical stimulation field representation (M) 1040, the stimulation configuration circuitry 962 may generate a control signal to the implantable stimulator 704 to deliver electrostimulation energy in accordance with the estimated stimulation setting (S). In response to the user rejection of the graphical stimulation field representation (M) 1040, the user may be prompted to modify the graphical stimulation field representation (M) 1040 via the GUI. FIG. 11B similarly illustrates a comparison between the graphical stimulation field representation (M′) 1150 and the user-provided graphical stimulation field representation (M) 1140, and a user selection to accept or reject the user-provided graphical stimulation field representation (M) 1140 based at least in part on the difference therebetween.

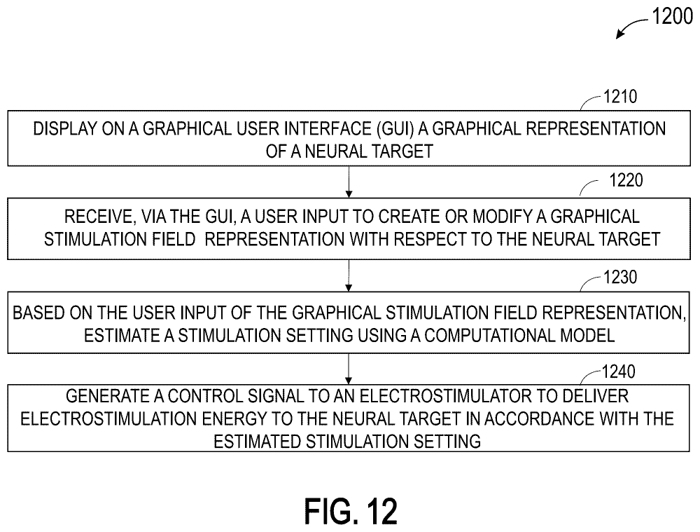

FIG. 12 is a flow chart illustrating and example method 1200 for programming an electrostimulator to provide electrostimulation to a patient. The method 1200 may be carried out using a programmer device such as the CP 630, the ETM 634, or the external programming device 802. In an example, the method 1200 may be used to program and provide closed-loop deep brain stimulation (DBS) at a brain target.

At step 1210, a graphical representation of a neural target of treatment for certain neurological disease or condition may be displayed on a graphical user interface (GUI) associated with the programmer device. The neural target, such as the “default” target neural structure 1020 as shown in FIG. 10A, may be a clinically acceptable therapeutic neural structure, such as Subthalamic Nucleus (STN) which is generally believed to be a DBS target for alleviating or rectifying motor symptoms of Parkinson's Disease (PD), among other movement disorders. The default target neural structure (e.g., STN) can be individually identified and localized from the patient during a pre-implantation imaging study.

At step 1220, a user input to create or modify a graphical stimulation field representation with respect to the neural target may be received from the GUI. In some examples, the user input to create or modify the graphical stimulation field representation may further be with respect to a base stimulation field model (SFM), such as SFM 1010 as shown in FIG. 10A. The base SFM defines a therapeutic region where the stimulation thereof would generally lead to clinically acceptable therapeutic effects (e.g., high therapeutic benefits such as symptom relief, and/or low side effects). The base SFM can be a “population-based” model in that can be based on implantation data obtained from a patient population. In an example, the base SFM can be an aggregation of several individual SFMs previously created and tested to lead to clinically acceptable therapeutic effects. The population-based, aggregated SFM and the patient-specific target neural structure may serve as reference volumes to assist the user in creating the graphical stimulation field representation. In some examples, information about the lead system being used for delivering electrostimulation and associated electrodes thereon, including lead location and active electrodes, may be graphically presented on the GUI.

The user may create or modify the graphical stimulation field representation using one or more user interface (UI) elements. The aggregated SFM, the default target neural structure, and the user-provided initial graphical stimulation field representation may be presented in a two-dimensional view (e.g., a cross-sectional view of a tissue volume) or a three-dimensional view. As described above with respect to FIGS. 10A-10B and 11A-11B, the user may provide an initial graphical stimulation field representation at a location spatially relative to the lead location or locations of one or more electrodes as displayed on the GUI. The initial graphical stimulation field representation has user-defined shape and size, and is located at a user-defined position with respect to the aggregated SFM and/or the patient-specific target neural structure. The initial graphical stimulation field representation represents a user designated target area intended to be stimulated in one example, a user designated avoidance region not intended to be stimulated in another example, or both in a further example.

The user may modify the initial graphical stimulation field representation using one or more UI control elements. The modification may include, for example, one or more of translating, rotating, resizing, flipping, or cropping of the initial graphical stimulation field representation or a portion thereof. FIGS. 10B and 11B illustrate examples of using a drag-and-drop UI control element to change the size and/or the shape of the initial graphical stimulation field representation, such as by mouse pressing and dragging an edge portion of the initial graphical stimulation field representation, and releasing the mouse when a desired size and shape is obtained.

At 1230, based on the received user input of the graphical stimulation field representation, a stimulation configuration (also referred to as a stimulation settings) may be estimated using a computational mode. The estimated stimulation configuration includes one or more stimulation parameters or electrode configurations that correspond to a coverage region (corresponding to the final test volume) substantially matching the received graphical stimulation field representation (corresponding to the target volume). In an example, the stimulation configuration may include information about fractionalization of stimulation current or energy across multiple active electrodes.

The estimation may include determining a stimulation test volume, activated by an application of the estimated stimulation setting, that substantially matches the received graphical stimulation field representation. In an example, the computational model includes an inverse modeling algorithm that automatically estimates, through an iterative process, a test volume of tissue in the patient, activated by the stimulation configuration, that substantially matches a target volume represented by the graphical stimulation field representation. The “substantial” match requirement is satisfied when a difference between the target volume and the “final” test volume to be resulted from delivery of neurostimulation using the estimated stimulation configuration falls within a specific margin, such that the iteration process can be terminated. The target volume can be refined by one or more iterations using the one or more clinical effects resulting from the test volume used in each iteration. Alternatively, the user may specify a test volume for each iteration. For example, the user may define a stimulation configuration (also referred to as a stimulation setting) or select one from stored stimulation configurations, and a test volume expected to be resulted from delivery of neurostimulation using the stimulation configuration can be determined. In one embodiment, the inverse modeling algorithm can be based on a SFM relating a stimulation configuration to a volume of activation. The stimulation configuration can be estimated using a library including data mapping volumes of activation to stimulation configurations and/or using an analytical derivation of the stimulation configuration that generates the stimulation volume. Further details of such inverse modeling algorithm are described in commonly owned U.S. patent application Ser. No. 15/902,163, entitled “Method and Apparatus For Clinical Effects-Based Targeting of Neurostimulation” and filed on Feb. 22, 2018, and U.S. patent application Ser. No. 16/219,551, entitled “Systems and Methods for Clinical Effect-Based Neurostimulation” and filed on Dec. 13, 2018, the description of which is hereby incorporated by reference in their entirety. The estimated stimulation configuration may be presented to the user on the GUI, as illustrated in FIGS. 10A-10B and 11A-11B.

At 1240, a control signal may be provided to the electrostimulator to deliver electrostimulation energy to the neural target in accordance with the estimated stimulation setting. In some examples, the user can choose to either send a notification (e.g., to the RC 45 or a smartphone with the patient) for a therapy reminder, or to automatically initiate or adjust neuromodulation therapy in accordance with the estimated stimulation setting.

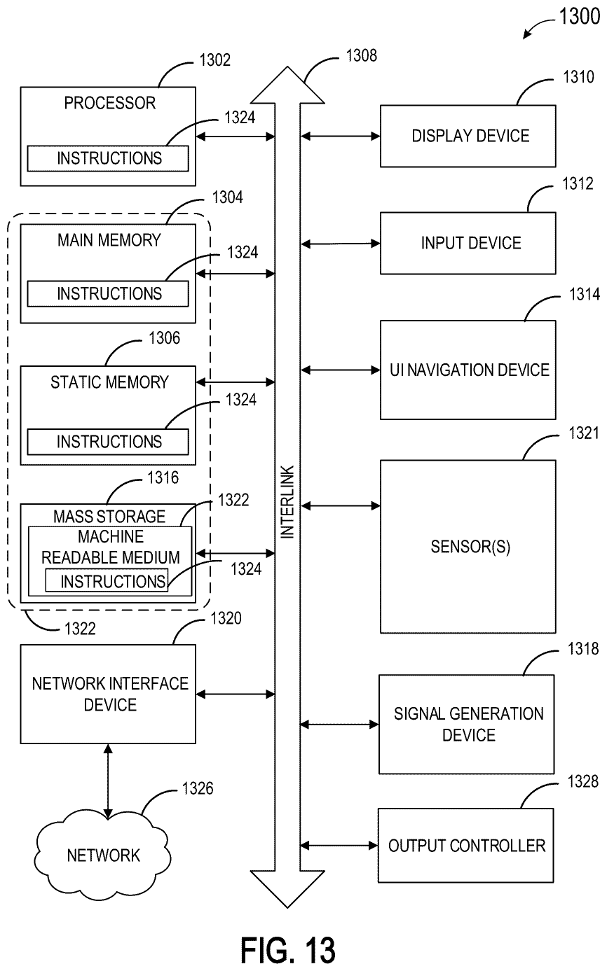

FIG. 13 illustrates generally a block diagram of an example machine 1300 upon which any one or more of the techniques (e.g., methodologies) discussed herein may perform. Portions of this description may apply to the computing framework of various portions of the neuromodulation device or the external programmer device.

In alternative examples, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1300 may include a hardware processor 1302 (e.g., a central processing unit (CPU), a graphics processing unit (GPU), a hardware processor core, algorithm specific ASIC, or any combination thereof), a main memory 1304 and a static memory 1306, some or all of which may communicate with each other via an interlink (e.g., bus) 1308. The machine 1300 may further include a display unit 1310 (e.g., a raster display, vector display, holographic display, etc.), an alphanumeric input device 1312 (e.g., a keyboard), and a user interface (UI) navigation device 1314 (e.g., a mouse). In an example, the display unit 1310, input device 1312 and UI navigation device 1314 may be a touch screen display. The machine 1300 may additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensors. The machine 1300 may include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1316 may include a machine-readable medium 1322 on which is stored one or more sets of data structures or instructions 1324 (e.g., software) embodying or utilized by any one or more of the techniques or functions described herein. The instructions 1324 may also reside, completely or at least partially, within the main memory 1304, within static memory 1306, or within the hardware processor 1302 during execution thereof by the machine 1300. In an example, one or any combination of the hardware processor 1302, the main memory 1304, the static memory 1306, or the storage device 1316 may constitute machine readable media.

While the machine-readable medium 1322 is illustrated as a single medium, the term “machine readable medium” may include a single medium or multiple media (e.g., a centralized or distributed database, and/or associated caches and servers) configured to store the one or more instructions 1324.

The term “machine readable medium” may include any medium that is capable of storing, encoding, or carrying instructions for execution by the machine 1300 and that cause the machine 1300 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1324 may further be transmitted or received over a communication network 1326 using a transmission medium via the network interface device 1320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communication network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1300, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A neuromodulation system, comprising:
an electrostimulator configured to provide electrostimulation to a neural target in a patient via at least one lead; and
a programmer device operable by a user to program the electrostimulator, the programmer device including:
a graphical user interface (GUI); and
a controller circuit configured to:
display on the GUI a graphical representation of the neural target;
receive, via the GUI, a user input to create or modify a graphical stimulation field representation with respect to the neural target;
based on the received user input of the graphical stimulation field representation, estimate a stimulation setting including one or more stimulation parameters or electrode configurations using a computational model, the estimation including determining a stimulation test volume, activated by an application of the estimated stimulation setting, that substantially matches the received graphical stimulation field representation; and
generate a control signal to the electrostimulator to deliver electrostimulation energy to the neural target in accordance with the estimated stimulation setting.

2. The neuromodulation system of claim 1, wherein the computational model includes an inverse modeling algorithm relating a stimulation volume to a stimulation setting.

3. The neuromodulation system of claim 1, wherein the controller circuit is configured to display on the GUI a graphical representation of a base stimulation field model,
wherein the user input to create or modify the graphical stimulation field representation is further with respect to the base stimulation field model.

4. The neuromodulation system of claim 1, wherein the GUI includes one or more user interface control elements operable by the user to provide the user input to create or modify the graphical stimulation field representation.

5. The neuromodulation system of claim 4, wherein the user input to create or modify the graphical stimulation field representation includes, via the one or more user interface control elements:
creating an initial graphical stimulation field representation; and
modifying the initial graphical stimulation field representation, including one or more of translating, rotating, resizing, flipping, or cropping of the initial graphical stimulation field representation or a portion thereof.

6. The neuromodulation system of claim 1, wherein the user input to create or modify the graphical stimulation field representation is spatially relative to the at least one lead,
wherein the estimated stimulation setting includes an electrode configuration specifying a selection of one or more active electrodes from the at least one lead and a fractionalization of electrical current flowing through the selected one or more active electrodes.

7. The neuromodulation system of claim 1, wherein the estimated stimulation setting includes the stimulation parameters including one or more of an amplitude, a pulse width, or a frequency of electrostimulation pulses.

8. The neuromodulation system of claim 1, wherein to receive the user input to create or modify the graphical stimulation field representation, the controller circuit is configured to create an initial graphical stimulation field representation based on a user selection of one or more of a stimulation target volume or a stimulation avoidance volume within or proximate the neural target.

9. The neuromodulation system of claim 1, wherein to receive the user input to create or modify the graphical stimulation field representation, the controller circuit is configured to create an initial graphical stimulation field representation based on a user input of a base stimulation setting.

10. The neuromodulation system of claim 1, wherein the controller circuit is further configured to:
determine a reference graphical stimulation field representation corresponding to the stimulation test volume; and
display on the GUI the reference graphical stimulation field representation overlaid upon the graphical stimulation field representation received as the user input.

11. The neuromodulation system of claim 10, wherein the controller circuit is further configured to:
present on the GUI an indication of a difference between the reference graphical stimulation field representation and the graphical stimulation field representation received as the user input; and
receive a user acceptance or rejection of the received graphical stimulation field representation.

12. The neuromodulation system of claim 11, wherein the controller circuit is configured to:
in response to the user acceptance of the received graphical stimulation field representation, generate the control signal to the electrostimulator to deliver electrostimulation energy in accordance with the estimated stimulation setting; and
in response to the user rejection of the received graphical stimulation field representation, prompt the user to further modify the received stimulation field model on the GUI.

13. A method of operating a programmer device to program an electrostimulator to deliver electrostimulation energy to a neural target in a patient via at least one lead, the method comprising:
displaying, on a graphical user interface (GUI) associated with the programmer device, a graphical representation of the neural target;
receiving, via the GUI, a user input to create or modify a graphical stimulation field representation with respect to the neural target;
based on the received user input of the graphical stimulation field representation, estimating a stimulation setting including one or more stimulation parameters or electrode configurations using a computational model, the estimation including determining a stimulation test volume, activated by an application of the estimated stimulation setting, that substantially matches the received graphical stimulation field representation; and
generating a control signal to the electrostimulator to deliver the electrostimulation energy to the neural target in accordance with the estimated stimulation setting.

14. The method of claim 13, wherein the computational model includes an inverse modeling algorithm relating a stimulation volume to a stimulation setting.

15. The method of claim 13, comprising displaying on the GUI a graphical representation of a base stimulation field model,
wherein the user input to create or modify the graphical stimulation field representation is further with respect to the base stimulation field model.

16. The method of claim 13, wherein the user input to create or modify the graphical stimulation field representation includes, via one or more user interface control elements in the GUI operable by the user:
creating an initial graphical stimulation field representation; and
modifying the initial graphical stimulation field representation, including one or more of translating, rotating, resizing, flipping, or cropping of the initial graphical stimulation field representation or a portion thereof.

17. The method of claim 13, wherein the user input to create or modify the graphical stimulation field representation is spatially relative to the at least one lead,
wherein the estimated stimulation setting includes an electrode configuration specifying a selection of one or more active electrodes from the at least one lead and a fractionalization of electrical current flowing through the selected one or more active electrodes.

18. The method of claim 13, wherein receiving the user input to create or modify the graphical stimulation field representation includes:
receiving a user selection of one or more of a stimulation target volume or a stimulation avoidance volume each within or proximate the neural target; and
automatically creating an initial graphical stimulation field representation based on the user selection of the one or more of a stimulation target volume or the stimulation avoidance volume.

19. The method of claim 13, wherein receiving the user input to create or modify the graphical stimulation field representation includes:
receiving a user input of a base stimulation setting; and
automatically creating an initial graphical stimulation field representation based on the base stimulation setting.

20. The method of claim 13, comprising:
determining a reference graphical stimulation field representation corresponding to the stimulation test volume;
presenting on the GUI an indication of a difference between the reference graphical stimulation field representation and the graphical stimulation field representation received as the user input;
receiving a user acceptance or rejection of the received graphical stimulation field representation;
in response to the user acceptance of the received graphical stimulation field representation, generating the control signal to the electrostimulator to deliver electrostimulation energy in accordance with the estimated stimulation setting; and
in response to the user rejection of the received graphical stimulation field representation, prompting the user to further modify the received stimulation field model on the GUI.
